(19) United States
(12) Patent Application Publication
(10) Pub. No.: US 2021/0400983 A1
(43) Pub. Date: Dec. 30, 2021

(54) ANTIMICROBIAL LATEX OR RUBBER PRODUCTS MADE USING A (NANO)COMPOSITE ZINC OXIDE, AND COMPOSITIONS AND METHODS FOR MAKING SUCH PRODUCTS

(71) Applicants: Raymond L. NIP, Bangkok (TH); Attaporn BANDIT, Samut Prakan (TH)

(72) Inventors: Raymond L. NIP, Bangkok (TH); Attaporn BANDIT, Samut Prakan (TH)

(73) Assignee: Global Chemical Co., Ltd., Samutprakarn (TH)

(21) Appl. No.: 16/911,589

(22) Filed: Jun. 25, 2020

(51) Int. Cl. A01N 59/16; C08K 3/22; C08J 9/12; A61L 31/04; A61L 31/16; A61L 31/14

(52) U.S. Cl. CPC A01N 59/16; C08K 3/22; C08J 9/125; A61L 31/049; A61L 31/16; A61L 31/143; A61L 2300/404; C08K 2003/2296; C08J 2307/02; C08J 2207/12; C08J 2203/10; A61L 2400/12; A61L 2300/102; C08K 2201/011

(57) ABSTRACT

A latex or natural rubber composition or formulation, latex or rubber products made using the same, and methods of preparing a vulcanized rubber and latex or natural rubber products are disclosed. The composition/formulation includes latex or natural rubber, 0.1-5 phr of a vulcanization activator, and 0.5-10 phr of one or more accelerators and/or additional activators. At least one accelerator and/or additional activator is a nanocomposite zinc oxide. The method includes. mixing a source of the latex or rubber with a vulcanization activator and one or more nanocomposite zinc oxides in a tank or vessel to form a latex or rubber formulation, dipping or at least partially immersing one or more molds or formers in the latex or rubber formulation to form a latex or rubber coating thereon, and curing the latex or rubber coating to vulcanize the latex or rubber coating. The vulcanized latex product is strong and has antimicrobial properties.

### FIELD OF THE INVENTION

The present invention generally relates to latex or natural rubber compositions and formulations, latex or rubber products made using the same, and methods of preparing vulcanized latex/rubber and making latex and natural rubber products having antimicrobial activity.

### DISCUSSION OF THE BACKGROUND

Zinc oxide coated particles and methods for their manufacture are known. Such particles are widely used in cosmetics and dry rubber goods like shoes, tire, belts and other products. Zinc oxide coated particles typically include a core material with zinc oxide coated on the outside. This material is also known as composite zinc oxide. Most core materials are relatively large, in the range of 2 to 5 microns. Adhesion of the zinc oxide to the core materials is usually not strong. Under some milling conditions such as those encountered in conventional jet milling, the zinc oxide can separate from the core material, especially when the core material is ground calcium carbonate. The core material acts as a filler, but does not always reinforce or support the zinc oxide, as the core material particle size can be too large and/or the binding of the zinc oxide to the core material may be too weak.

U.S. Pat. No. 7,635,729 discloses methods of coating particles with zinc oxide and/or zinc carbonate using lime (calcium oxide and/or calcium hydroxide). According to embodiments of these methods, calcium hydroxide (from lime) can be made into nanoscale particles (e.g., from 10 to 100 nanometers). During the coating process, the core material (calcium hydroxide) can react with carbon dioxide to form very fine calcium carbonate particles. At the same time, the calcium hydroxide and/or water (e.g., a source of hydroxide ions) can react with zinc ions (e.g., zinc oxide) to form zinc hydroxide. In turn, the zinc hydroxide can react with excess carbon dioxide to form zinc carbonate. As all of the reactions are chemical in nature, the reaction product (e.g., from zinc ammonia carbonate complex) adheres tightly to the core materials to form fine particle sized zinc carbonate and/or zinc hydroxide or zinc hydroxyl carbonate. By the methods disclosed in U.S. Pat. No. 7,635,729, the resulting zinc oxide (e.g., after heating and/or drying) and/or zinc carbonate coating adheres well to the core material (calcium hydroxide and/or calcium carbonate).

The calcium hydroxide and/or calcium carbonate core material disclosed in U.S. Pat. No. 7,635,729 may have a very small size. For example, precipitated calcium carbonate may have a size in the range of 10-150 nanometers, which is equal to a BET surface area of 10-30 m²/g or more. Such calcium carbonate is very reinforcing in dry rubber compounds, especially tires. It can be considered as a nanofiller. For tire treads, it provides very good abrasion resistance.

Latex gloves are used mostly in the food industry and in the medical field. Prior to use, such gloves need to be clean and free of bacteria, fungi and viruses. It is desirable for the gloves to be able to inhibit or kill such organisms (i.e., to be “antimicrobial”), especially during the recent coronavirus pandemic, which is spreading rapidly around the world and which, as of June 2020, does not have a vaccine or a generally-recognized safe and effective therapy.

The conventional methods to make gloves antimicrobial is to include a biocide in the latex compound. The U.S. Environmental Protection Agency (EPA) defines “biocide” as a diverse group of poisonous substances including preservatives, insecticides, disinfectants, and pesticides used for the control of organisms that are harmful to humans or animals. However, many bacteria and fungi have developed resistance to antibiotics and other biocides.

Zinc oxide is used in many rubber vulcanization processes as an activator. In latex gloves, its dosage is only 0.5 to 1 part per hundred parts of rubber (phr) by weight. It is usually made into a 50 wt. % water slurry, then added into the latex before the mold is dipped into the latex and sent to an oven for vulcanization. The latex can be natural or synthetic latex.

In recent years, it was discovered that nano-sized zinc oxide can have antibacterial, antifungal, and antiviral properties. However, for many applications, an amount of zinc oxide in excess of 1 phr is needed for antimicrobial activity. The excess zinc oxide makes the latex product hard and brittle, which is undesirable for latex (which is otherwise highly elastic).

It was also discovered that composite zinc oxide can inhibit and kill microorganisms (CN103651569A) and make rubber gloves stronger, more stretchable, and more aging-resistant. At the same time, the net amount of zinc oxide in the latex formulation for making gloves is reduced, relative to zinc oxide alone. This reduction is important for environmental reasons, as zinc may be harmful to fish and other aquatic organisms.

However, large-sized fillers may result in pin holes in the gloves or other latex product. This is especially undesirable for condoms. This may be one reason why the composite zinc oxide disclosed in CN103651569A is not used in the rubber/latex glove industry

This “Discussion of the Background” section is provided for background information only. The statements in this “Discussion of the Background” are not an admission that the subject matter disclosed in this “Discussion of the Background” section constitutes prior art to the present disclosure, and no part of this “Discussion of the Background” section may be used as an admission that any part of this application, including this “Discussion of the Background” section, constitutes prior art to the present disclosure.

### SUMMARY OF THE INVENTION

Embodiments of the present invention relate to compositions containing zinc oxide-coated particles or composite zinc oxide, and methods of making and using the same, in which the zinc oxide functions, e.g., as a biocide. A need is still felt for latex products, especially thin latex products such as gloves and condoms, with antimicrobial properties and a relatively low toxicity, but which otherwise retains most or all of its strength, elasticity and non-permeability. The composite zinc oxide disclosed in U.S. Pat. No. 7,636,729 is an acceptable accelerator in the manufacture of latex gloves, as the composite zinc oxide particles are relatively small, and the gloves are very thin. However, doubts about the ability of the composite zinc oxide disclosed in U.S. Pat. No. 7,636,729 to provide commercially acceptable thin latex products may have existed prior to the present invention, especially if the zinc oxide is coated on ground calcium carbonate instead of precipitated calcium carbonate or coprecipitated calcium carbonate and lime, even though the composite zinc oxide disclosed in U.S. Pat. No. 7,636,729 may have been known to provide rubber products with an acceptable modulus of elasticity, tear strength, and aging properties. The present invention is based at least in part on the discovery that nanocomposite zinc oxide made by a method disclosed in U.S. Pat. No. 7,636,729 has biocidal properties, in addition to making the finished latex/rubber product better and stronger. With nanocomposite zinc oxide inhibiting or controlling the growth of harmful organisms, it can replace conventional biocides in latex/rubber gloves and other latex/rubber products. The composite zinc oxide disclosed in U.S. Pat. No. 7,636,729 is not toxic or harmful to humans.

In one aspect, the present invention provides a composition or formulation, comprising latex or natural rubber, a vulcanization activator in an amount of 0.1-5 parts per hundred parts of the latex or natural rubber (phr), and one or more accelerators and/or additional activators including a nanocomposite zinc oxide in an amount of 0.5-10 phr.

The nanocomposite zinc oxide may comprise zinc oxide-coated calcium carbonate having an average primary particle size of 100 nm or less, a particle size distribution in which ≥50% of the particles have a size of 100 nm or less, and/or a BET surface area of ≥10 m²/g. The accelerator(s) and/or additional activator(s) may further comprise zinc oxide and/or a composite zinc oxide, in addition to a nanocomposite zinc oxide.

Typically, the composition or formulation further comprises water. In various embodiments, the water is present in an amount of 50-100 phr, although the composition or formulation is not limited to this range.

In further embodiments, the composition or formulation further comprises a release agent, an antioxidant, a stabilizer, a pigment, a dispersion agent, a plasticizer and/or one or more fillers. The release agent may be present in an amount of 0.1-10 phr (e.g., 0.1-2 phr). The antioxidant may be present in an amount of 0.1-3 phr. The stabilizer may be present in an amount of 0.1-5 phr. The pigment may be present in an amount of 0.5-20 phr. The dispersion agent may be present in an amount of 0.1-5 phr. The plasticizer may be conventional, and may be present in an amount of 0.1-10 phr. The filler(s), which may be selected from the core materials disclosed herein for the composite, (nano)composite and nanocomposite zinc oxide or other conventional latex or rubber fillers, may be present in an amount of 1-30 phr.

Another aspect of the present invention relates to a latex or rubber product made from the present composition or formulation. For example, the latex or rubber product may have (i) a tensile strength of at least 30 MPa or a tear strength of at least 40 kN/m, and (ii) an antimicrobial activity greater than that of an otherwise identical latex or rubber product made from an otherwise identical composition or formulation substituting an identical amount of a French process zinc oxide for the nanocomposite zinc oxide. Alternatively or additionally, the latex or rubber product may have a modulus of elasticity at 300% or greater elongation that is greater than that of the otherwise identical latex or rubber product (made from the otherwise identical composition or formulation substituting the identical amount of the French process zinc oxide for the [nano]composite or nanocomposite zinc oxide). In some embodiments, the antimicrobial activity of the latex or rubber product is greater than that of an otherwise identical latex or rubber product made from an otherwise identical composition or formulation substituting an identical amount of a nanoparticulate zinc oxide for the nanocomposite zinc oxide.

The latex or rubber product may be or comprise a glove, condom, or balloon. In such cases, the latex or rubber product may have a thickness of 0.05-0.5 mm.

Alternatively, the latex or rubber product may further comprise a fabric. In such a case, the latex or rubber may be incorporated into the fabric, and the product may be or comprise hospital bedding, a pillow case, a non-latex glove, clothing, boots or shoes. When used as personal protective equipment (PPE) in a hospital or other medical setting, such products (e.g. including the present latex or rubber formulation or composition incorporated into a fabric) can reduce the probability of cross-contamination of patients with microbes.

In some embodiments, the latex or rubber product may be or comprise a foam rubber product, such as a mattress, carpet backing, a pillow, or other latex foam product. Such products may be made from the present latex or rubber formulation or composition, using conventional methods or processes for making foam rubber or latex foam (e.g., by adding a blowing agent, such as a gas [e.g., a hydrofluorocarbon having 1-3 carbon atoms] or a chemical that produces a gas [e.g., a carbonate, bicarbonate or azide salt], to create a mass of small bubbles in the liquid latex composition or formulation). The liquid latex composition or formulation for making foam rubber or latex foam may further comprise one or more polyols, polyisocyanates, flame retardants, fillers, and/or pigments/colorants.

Another aspect of the present invention concerns a method of preparing a vulcanized latex or rubber, comprising mixing a source of the latex or rubber with a vulcanization activator or vulcanizing agent, one or more nanocomposite zinc oxides, and optionally one or more additional accelerators and/or activators, pigments, stabilizers, release agents, and/or antioxidants in a tank or vessel to form a latex or rubber formulation; dipping or at least partially immersing the mold(s) or former(s) in the latex or rubber formulation to form a latex or rubber coating on the mold(s) or former(s); and curing the latex or rubber coating to vulcanize the latex or rubber coating.

In some embodiments, the method may further comprise (i) washing one or more molds or formers in water and/or an aqueous chlorine-based solution prior to dipping or at least partially immersing the one or more molds or formers in the latex or rubber formulation, and/or (ii) prior to curing the latex or rubber coating, immersing the latex or rubber coating in a mixture of water and a chlorine source. Such washing may be conducted at a temperature and for a length of time sufficient to remove residual latex, rubber and/or components of the latex or rubber formulation from the mold(s) or former(s), and such immersing may be conducted at a temperature and for a length of time sufficient to remove residual latex proteins and non-rubber chemicals and/or reduce the severity of any allergic reactions to the latex. In either the washing or the immersing, the temperature of the water, aqueous chlorine-based solution or mixture of water and chlorine source may be from 20° C. to 80° C.

In other or further embodiments, the method may further comprise drying the latex or rubber coating (which may still be on the mold[s] or former[s]). The latex or rubber coating may be dried prior to or during curing. In further embodiments, curing the latex or rubber coating may comprise heating the curable rubber composition at a temperature of at least 80° C. for at least 10 minutes. The curing temperature may be any temperature or temperature range of at least 100° C. (for example, 80-130° C., 100° C. to 150° C., etc.), and the curing time may be, for example, from 30 minutes to 8 hours (or any length of time or range of lengths of time of at least 10 minutes).

The present invention also relates at least in part to a method of making a latex or rubber product, comprising the present method of preparing a vulcanized latex or rubber, and releasing the cured latex or rubber coating from the mold(s) or former(s) to produce the latex or rubber product. Similar to the present latex or rubber product described above, the latex or rubber product produced by the present method has antimicrobial activity and may have an elongation at break over 700%. Alternatively or additionally, the latex or rubber product produced by the present method may have a tensile strength, a tear strength, and/or a modulus of elasticity that are equal to or better than an otherwise identical latex or rubber product made from an otherwise identical latex or rubber formulation containing an amount of zinc oxide identical to an amount of the nanocomposite zinc oxide(s) in the present latex or rubber formulation. The antimicrobial activity of the latex or rubber product produced by the present method may be equal to or better than an otherwise identical latex or rubber product made from the otherwise identical latex or rubber formulation containing the amount of zinc oxide (e.g., nanoparticulate ZnO) identical to the amount of the nanocomposite zinc oxide(s) in the present latex or rubber formulation.

These and other advantages of the present invention will become readily apparent from the detailed description of various embodiments below.

### DETAILED DESCRIPTION

Reference will now be made in detail to various embodiments of the invention. While the invention will be described in conjunction with the disclosed embodiments, it will be understood that they are not intended to limit the invention. On the contrary, the invention is intended to cover alternatives, modifications and equivalents that may be included within the spirit and scope of the invention as defined by the appended claims. Furthermore, in the following detailed description of the present invention, numerous specific details are set forth in order to provide a thorough understanding of the present invention. However, it will be readily apparent that the present invention may be practiced without these specific details. In other instances, well-known methods, procedures and/or components have not been described in detail so as not to unnecessarily obscure aspects of the present invention. Furthermore, it should be understood that the possible permutations and combinations described herein are not meant to limit the invention. Specifically, variations that are not inconsistent may be mixed and matched as desired.

For the sake of convenience and simplicity, a “composite zinc oxide” refers to a substance comprising a core material and/or particle and a zinc oxide and/or zinc carbonate coating thereon, containing from 1% to 80% by weight of zinc oxide and/or zinc carbonate. The core material may comprise a clay, talc, mica, silica, calcium hydroxide, calcium oxide and/or calcium carbonate. A “nanocomposite zinc oxide” refers to a composite zinc oxide in which both the core material/particle and the zinc oxide/zinc carbonate coating have an average primary particle size of 100 nm or less (e.g., 2-100 nm), or a particle size distribution in which ≥50% of the particles have a particle size of 100 nm or less. Alternatively, a nanocomposite zinc oxide may have a BET surface area of ≥20 m²/g or any other minimum value ≥20 m²/g (e.g., ≥30 m²/g, ≥100 m²/g, etc.). A “primary” particle size refers to the size of individual or non-agglomerated particle. A nanocomposite zinc oxide may be referred to as “double play nanoparticles,” as both the core and the coating are nanoparticles. If the core is a nanoparticle having an average size of, e.g., 100 nm, the coating particles (i.e., of zinc oxide) are likely to be even smaller nanoparticles, having an average size of, e.g., 20 to 40 nm. However, as shown in this example, the combined core and coating particles in the nanocomposite zinc oxide may have a total particle size >100 nm (e.g., up to 500 nm, or any other maximum value >100 nm and <500 nm, such as 200 nm). The core may also act as a catalyst carrier, helping to disperse the zinc oxide (e.g., in the coating) and reduce its agglomeration.

A “nanoparticulate zinc oxide,” “nanoscale zinc oxide” or “nano zinc oxide” refers to a zinc oxide having a primary particle size of less than 100 nm. However, nanoparticles (particularly of ZnO) tend to agglomerate or stick together, and the particle size of ZnO is often considered to be the size of the agglomerated particles, which is typically 80 to 3,000 nanometers. An “HC grade” of zinc oxide is an active zinc oxide with a surface area of 20-30 square meters per gram, an agglomerated particle size of about 2 μm or more, and a purity of at least 99%. It is produced commercially by Global Chemical Co. Ltd., Samut Prakarn, Thailand and is also a nano zinc oxide with a primary particle size of about 50 nm.

Natural and synthetic latex products include gloves, condoms, balloons, and certain foam rubber products, such as pillows and mattresses. Such products should be (and for certain applications must be) clean and free of bacteria, fungi and viruses. Consequently, it is desirable to provide latex and other rubber products with antibacterial, antifungal and/or antiviral properties.

The nanocomposite zinc oxides contemplated for use in the present invention do not render the latex products brittle or less elastic at the same or higher dosages than French process zinc oxide. Its lower cost and lower zinc discharge to the environment (e.g., as compared to ZnO) are additional advantages.

Exemplary Nanocomposite Zinc Oxides and Exemplary Methods of Making the Same

Composite zinc oxides, nanocomposite zinc oxides, and methods of making the same are disclosed in U.S. Pat. No. 7,636,729, the relevant portions of which are incorporated herein by reference. In composite zinc oxides, a coating comprising an oxide and/or carbonate of zinc is formed on one or more inorganic substrate particles such as clay, talc, silica, calcium carbonate, calcium oxide, and the like, which are mostly not nanoparticles, but on which the deposited ZnO coating can take the form of nanoparticles. The zinc material is precipitated onto the substrate particles from an aqueous zinc-containing solution and a slurry of the substrate particles. The zinc-containing solution is generally basic. Most conveniently, it is obtained by dissolving zinc oxide and/or other zinc bearing materials like zinc ash, roasted zinc sulfide ore, etc., in an aqueous solution comprising an ammonia source (e.g., ammonium hydroxide) and a carbon dioxide source (e.g., carbon dioxide, ammonium carbonate, etc.) in a vessel or reactor to form a zinc ammonia carbonate complex (Zn[NH₃]₄CO₃) solution. French process zinc oxide is one example of a zinc oxide suitable for use in this process. Other zinc sources like zinc ash can also be substituted, but purification of the zinc ammonia carbonate complex solution (e.g., according to U.S. Pat. No. 5,204,084, the relevant portions of which are incorporated herein by reference) may then be desirable. The weight percent of ammonia in the solution can be from 1% to about 20%. The amount of zinc dissolved in the solution may be (and preferably is) from 1 to 15% by weight. The molar ratio of ammonia to carbon dioxide in the solution preferably is from about 10:1 to 2:1. The zinc ammonia carbonate complex solution desirably is formed at a temperature in the range of from about 20° C. to about 80° C.

The zinc ammonia carbonate complex solution can optionally be replaced with one or more aqueous solutions of other zinc ammine salts like zinc ammine chloride or zinc ammine sulfate, which can be formed in situ from the corresponding zinc salt and an ammonia source (e.g., ammonia or ammonium hydroxide). The zinc source may also comprise a zinc salt (e.g., of the formula ZnX₂, wherein X is a halogen or monoanionic group, or X₂ taken together is a dianionic group such as sulfate or carbonate).

A wide range of substrate particles may be used, such as clay, talc, silica, mica, silicon nitride, silicon carbide, activated carbon, carbon black, combinations of these and the like. The particles may be platy and/or inorganic. Suitable clays can include commercially available air-floated clays (e.g., Clay S-40 or C-400 from Siam Soil Co. Ltd., Bangkok, Thailand). Other examples of commercially available substrate particles include talc from Liaoning Jiayi Metal & Minerals Co. Ltd., Dalian, China, and precipitated silica from United Silica (Siam) Ltd., Rayong, Thailand.

The relative amounts of substrate particle slurry and zinc-containing solution are selected so that the resultant composite particles may include from about 1.6 to about 20 weight percent zinc (generally corresponding to about 2 to 25 weight percent zinc oxide). In applications in which the resultant composite particles may be used in transparent or translucent rubber products or polymeric products with silica fillers (or just as a rubber activator), the resultant composite particles may include up to 70 weight percent of zinc oxide (about 56 weight % zinc), typically about 0.5 to 65 wt. %, even more typically 5 to about 65 weight percent zinc oxide (about 4 to about 52 weight % zinc).

The substrate particles and the zinc (e.g., zinc ammonia carbonate complex) solution are mixed in a reaction vessel with good agitation. The reaction may occur with heating and/or by adding an acid. Heating may help drive off the ammonia and cause zinc-containing material (e.g., zinc oxide and/or carbonate) to precipitate onto the substrate particles. Carbon dioxide will also come out of the reaction mixture if excess carbon dioxide is present. A suitable temperature for the reaction may be from 80° C. to 150° C. (e.g., 100° C.), and the pressure may be from 1 psi to 100 psi (e.g., atmospheric pressure). The reaction may be complete when substantially all ammonia is liberated. At this stage, most or all of the zinc will also be precipitated, thereby forming the nanocomposite zinc oxide. After most or all of the ammonia is driven out, the pH of the slurry/reaction mixture is around 7 to 9. One way to determine an end point of the reaction is when the pH is from 7 to 8. The zinc coating material precipitated onto the substrate particles is mostly in the form of zinc carbonate, zinc oxide and/or zinc hydroxide. It may be also known as basic zinc carbonate, with a chemical formula Zn(OH)ₓ(CO₃)_{y}, where 0<x<2, 0<y<1, and 2x+y=1.

Optionally, in place of or in addition to heating, the zinc carbonate/zinc hydroxide/zinc oxide coated substrate particles can also be formed by addition of acid(s) such as sulfuric acid, hydrochloric acid, carbon dioxide (carbonic acid), etc., to lower the pH of the initial slurry (formed using a zinc ammine or zinc ammonia carbonate complex) to around 7 to 8.

After completion of the reaction, the slurry is filtered and washed (e.g., with water). The filter cake is then dried and calcined. The drying/calcining temperature can vary from 110° C. to 800° C. If the initial precipitate is zinc oxide and/or zinc hydroxide, a drying temperature of 110-300° C. produces a zinc oxide-coated composite particle. If the initial precipitate is mostly zinc carbonate, a drying temperature over 300° C. can decompose the zinc carbonate into zinc oxide. After drying, the dried powder may then be milled (e.g., to where at least 99.9% of the composite zinc oxide is below 325 mesh). The zinc oxide content of the resultant composite zinc oxide is anywhere from 1% to 80% by weight (or any value or range of values therein), depending on the ratio of zinc to particles in the initial slurry.

In another embodiment, nanocomposite zinc oxide may be formed by a co-precipitation technique. For example, zinc-containing material and calcium carbonate may be co-precipitated from a mixture comprising an aqueous zinc containing solution (e.g., a zinc ammonia carbonate solution as described above) and milky lime under heating. The precipitate is carbonated with carbon dioxide until substantially all of the lime is converted to calcium carbonate and substantially all of the zinc is precipitated. The core calcium carbonate material is usually nanoscale calcium carbonate, having an average particle size below 100 nm and/or a BET surface area equal to or greater than 22 m²/g. In general, the BET surface area of particles is related to the average particle size. The bigger the surface area, the smaller the particles. The formula for nanoscale calcium carbonate is S=2.21/d, where S is the BET surface area in m²/g, and d is the diameter of the particle in microns (1 micron=1,000 nm). Other materials have a different relationship between BET surface area and average particle size, as the density of other materials may be different from precipitated calcium carbonate. The porosity of the other materials, if different from precipitated calcium carbonate, may also have an effect on the relationship between BET surface area and average particle size of the material. The resulting nanocomposite material may be washed, dried, and calcined to provide a nanocomposite zinc oxide (e.g., a nanoscale zinc oxide coating on precipitated calcium carbonate).

Co-precipitated zinc oxide and calcium carbonate has advantages. Precipitated calcium carbonate, which may be prepared by reacting lime slurry with carbon dioxide, has a larger surface area and smaller particle sizes than ground calcium carbonate. The particle size of the calcium carbonate may be important, especially for rubber products. In the co-precipitation technique disclosed herein, one can produce precipitated calcium carbonate and coat it with zinc oxide at the same time. It is beneficial to coat a small amount of zinc oxide on very fine calcium carbonate particle and use the product as a filler, an activator, and an anti-microbial agent. The composite structure helps with the dispersion of zinc oxide while adding two functionalities into the rubber in one step. Coating zinc oxide on smaller calcium carbonate particles helps increase the surface area of the zinc oxide-coated calcium carbonate and helps with its dispersion. In rubber compounding, this means better vulcanization and savings on the cost of the zinc activator. Precipitated calcium carbonate also has better reactivity with acids. This means that the composite particle will better absorb and neutralize any acid produced when rubber articles age, thereby facilitating the aging resistance of rubber products.

More specifically, a milky lime (e.g., lime is slaked with water) is added to the basic (i.e., pH>9) zinc ammonia carbonate complex solution with heavy agitation (stirring). The zinc ammonia carbonate complex solution can optionally be replaced with a solution of a zinc ammine salt, like zinc ammine chloride, zinc ammine sulfate, or a mixture thereof. The milky lime can include from 1 to 15% of CaO on a theoretical basis (although the milky lime slurry actually includes calcium hydroxide). The resultant admixture is heated, for example at a temperature of 80° C. to 150° C., and at a pressure of from vacuum (e.g., 1 psi) to 100 psi. At atmospheric pressure, heating the solution to boiling (e.g., 100° C.) may be desirable.

Heating the mixture of milky lime and the zinc-containing solution liberates ammonia from the mixture. Carbon dioxide also evolves if excess carbon dioxide is present. The reaction is generally deemed to be complete when substantially all ammonia is liberated. At this stage, most or all of the zinc (e.g., from the zinc ammonia complex or zinc ammine solution) is precipitated onto the calcium hydroxide.

After evaporation of the ammonia and precipitation of the zinc are complete, the pH of the reacted slurry should be in the range of 7 to 10, although it can be higher than 10 in some cases. When the end point pH is 8 or higher (e.g., about 9 or higher), the precipitated zinc is substantially in the form of zinc oxide or zinc hydroxide. However, if the amount of carbon dioxide in the original solution is large, most or all of the lime may be converted to calcium carbonate during precipitation, and the end point pH will generally be 9 or lower (e.g., about 8 or lower). If enough carbon dioxide is present in the zinc ammonia carbonate complex solution, some or most of the zinc may precipitate as zinc carbonate.

After most or all of the ammonia is driven off, the mixture is cooled (e.g., to 50° C. or below), and carbon dioxide is added (e.g., by bubbling) to carbonate any excess lime (e.g., convert it into calcium carbonate). The precipitate is carbonated with carbon dioxide to a pH of 7 or below, at pressure from atmospheric pressure to 30 psi.

The resulting slurry is then filtered, washed, dried and calcined as described above, although the maximum drying/calcining temperature may be 600° C. After drying, the dried nanocomposite zinc oxide (nanoscale zinc oxide on precipitated calcium carbonate) is then milled as described above. The zinc oxide content of the resultant composite particles is from 1% to 80% by weight, or any value or range of values therein.

In the methods of preparing nanocomposite zinc oxide on calcium carbonate core particles, several competing reactions may be occurring in the formation of zinc carbonate or basic zinc carbonate. For example, driving out the ammonia from a zinc ammonia carbonate complex solution precipitates zinc carbonate. At the same time, Zn²⁺ ions may react with the lime slurry to form zinc hydroxide, and the zinc hydroxide may react with carbon dioxide (or with carbonate or bicarbonate ions) to form zinc carbonate.

The lime particles in the slurry may be relatively large. It is estimated that, under conditions described herein or representative of those described herein, 40-50% of the lime particles react with zinc ions to form zinc hydroxide, and the remainder reacts with carbonate ions (or CO₂ plus water) to form calcium carbonate. The zinc hydroxide may or may not react with carbon dioxide to form zinc carbonate under such conditions. After washing, drying, and calcining, the zinc oxide adhesion to the calcium carbonate is much stronger than in a similar method of coating ground calcium carbonate with zinc oxide (see, e.g., U.S. Pat. No. 4,207,377, which does not form nanocomposite zinc oxide). For example, during jet milling of the nanocomposite zinc oxide formed by the processes described herein, the ZnO nanoparticles stick tightly to the calcium carbonate core particle before and after jet milling. In contrast, the zinc oxide separates easily from the ground calcium carbonate in the composite zinc oxide made in accordance with the disclosure of U.S. Pat. No. 4,207,377.

Exemplary Methods of Making Latex Gloves and Other Products

The present invention relates in part to a method of making natural rubber and/or latex gloves and other products.

In certain embodiments, the method of making natural rubber and/or latex gloves uses ceramic or aluminum hand-shaped molds or formers that may be first washed extensively in hot water and/or a chlorine-based solution (e.g., bleach or other metal hypochlorite salt) to ensure there is no residue on the molds from previous processing. Next, the molds or formers may be suspended on a continuous moving chain and dipped into a solution (e.g., an aqueous solution) of mixture of a coagulant (e.g., calcium nitrate) and a release agent (e.g., calcium carbonate). When the components in the latex formulation/composition (see, e.g., the section entitled, “Exemplary Latex Compositions/Formulations Containing Composite Zinc Oxide” below) include one or both of these components, this step can be omitted.

Prior to the molds being immersed in a tank or other vessel containing the latex formulation/composition, the source of the latex (e.g., concentrated natural latex, which is widely commercially available) is mixed with processing components including a vulcanization activator or vulcanizing agent such as sulfur, one or more nanocomposite zinc oxides (alone or with zinc oxide), one or more additional accelerators, pigments, stabilizers, a de-webbing or release agent, and antioxidants. The latex matures for 24 to 36 hours to become a compound ready for dipping or immersion.

After drying, the molds are dipped or partially immersed (e.g., to a predetermined depth) into the latex formulation. The thickness of the gloves may be determined by the duration of the dip/immersion and the viscosity of the latex slurry. The freshly molded gloves may then be immersed in a mixture of hot water and chlorine (e.g., bleach or other metal hypochlorite salt), which can remove residual latex proteins and non-rubber chemicals to reduce the severity of any allergic reactions to the latex.

The gloves are then dried and cured, which is where the vulcanization process converts the gloves to an elastic state by reacting the rubber molecules in the latex with certain chemicals/additives in the latex formulation/composition, to give the gloves elasticity reduce the likelihood of tearing.

After drying, the gloves may be rinsed again to remove more latex proteins, and the cuffs of the gloves may be beaded (e.g., rolled) to make them easier for users to put on and take off After an optional dip into or spray with cornstarch (or other release/anti-adhesion agent) and/or an optional final drying, the finished gloves are removed from the molds or formers, manually (e.g., by hand) or using pneumatic air jets directed at or along the surface of the mold/former. The gloves may then be tumbled (e.g., in a heated tumbler) to remove any excess or remaining release/anti-adhesion agent from the finished gloves.

The molds are given another thorough chemical (e.g., aqueous bleach or hypochlorite salt) wash and rinse, and the process begins anew. Condoms and other thin latex products may be made with a similar or identical method/process.

In specific embodiments, most or all components in the formulation/composition (see, e.g., the section entitled, “Exemplary Latex Compositions/Formulations Containing Composite Zinc Oxide” below) except the latex may first be made into a dispersion, usually with one or more dispersion agents and/or other additives. The dispersion is placed in a tank, and the remaining ingredient(s) (e.g., concentrated natural latex) is/are added with good agitation (e.g., mechanical stirring). Thereafter, a mold with one or more mold release agents thereon is dipped into the latex mixture/suspension. The mold is removed when enough latex adheres to the mold. The latex gloves (on a plurality of the molds) are then dried and cured in air in an oven at a predetermined temperature (e.g., 100° C. to 250° C., or any temperature or range of temperatures therein, such as 120-220° C. or 130-185° C.) for a length of time of from 10 minutes to 8 hours (or any length of time or range of time lengths therein, such as about one hour), although the invention is not limited to these temperature ranges or lengths of drying/curing time. The molds with the gloves thereon are removed from the oven, and the latex gloves are released from the molds. The gloves may be dusted with a powdery material, such as zinc stearate, talc, a starch, etc., so that the gloves do not adhere to each other. The gloves may be examined for pin holes or other defects, then they are ready for packaging and shipment.

The gloves should have certain thickness, strength (e.g., tear strength), modulus (e.g., of elasticity), and elongation at break, which is usually over 700% (a somewhat universally recognized property of commercially acceptable latex gloves).

Exemplary Latex Compositions/Formulations Containing Composite Zinc Oxide

Compositions and formulations useful in the present invention include a source of latex or natural rubber, a vulcanization activator or vulcanizing agent, the nanocomposite zinc oxide (e.g., as an accelerator/additional activator and an antimicrobial agent), and water. Optional components in such compositions and formulations include stabilizers, antioxidants, other accelerators, pigments, release agents, dispersion agents, plasticizers, fillers, etc.

The most common source of latex or natural rubber is concentrated natural latex (e.g., having a 60% dry rubber content [DRC]), although the invention is not limited to this source. The amounts of the other components in the composition and/or formulation are discussed in amounts relative to 100 parts by weight of dry latex or natural rubber in the composition and/or formulation.

Stabilizers include alkali and alkaline earth soaps, such as sodium, potassium or calcium C₁₂-C₂₄ saturated, monounsaturated or polyunsaturated carboxylate salts (such as sodium or potassium laurate, myristate, palmitate, stearate, ricinoleate, oleate, linoleate and/or linolenate). The alkali or alkaline earth soap may be added as a solution or emulsion containing 5-50 wt. % (e.g., 20 wt. % or any wt. % or wt. % range therein) of the alkali or alkaline earth soap, in an amount providing 0.1-5 phr of the alkali or alkaline earth soap.

The vulcanization activator or vulcanizing agent may include sulfur. The vulcanization activator or vulcanizing agent may be added to the formulation or composition as a dispersion, for example in water, at a mass loading and/or weight percentage of 20-80% (e.g., 50% or any other value or range of values therein) in the dispersion. The vulcanization activator or vulcanizing agent may be added to the latex composition/formulation in an amount of 0.1-5 phr (or any amount or range of amounts therein).

Accelerators may include nanocomposite zinc oxides as disclosed herein, alone or together with one or more other accelerator(s) and/or additional activators, such as zinc oxide and/or one or more zinc dialkyldithiocarbamates such as zinc diethyldithiocarbamate (ZDEC), zinc dimethyldithiocarbamate and zinc dibutyldithiocarbamate, zinc alkylenedithiocarbamates such as zinc ethylenebisdithiocarbamate, zinc diaryl- and diaralkyldithiocarbamates such as zinc dibenzyldithiocarbamate, zinc mercaptoarenethiazolates such as zinc 2-mercaptobenzothiazolate (ZMBT). A preferred nanocomposite zinc oxide is nanocomposite ZnO NC236, a precipitated calcium carbonate coated with active ZnO and containing about 60% by weight of ZnO, commercially available from Global Chemical Co. Ltd., Samut Prakarn, Thailand. The zinc oxide may be a French process ZnO (e.g., commercially available from Univenture Public Ltd., Thailand), as HC grade, nano-ZnO commercially available from Global Chemical Co. Ltd. French process zinc oxide generally has a primary particle size of about 800 nm to 2,000 nm and a BET surface area of around 2-8 m²/g. The accelerator(s) may be added to the formulation or composition as a dispersion, for example in water, at a mass loading and/or weight percentage of 20-80% (e.g., 50% or any other value or range of values therein) in the dispersion. The accelerator(s) may be added to the latex composition/formulation in an amount of 0.5-10 phr (or any amount or range of amounts therein).

Antioxidants may include phenols, arenes, aryl amines, certain amino acids (e.g., cysteine/cystine, tyrosine, asparagine, phenylalanine, alanine), etc. A preferred antioxidant is a hindered phenol-type antioxidant (e.g., commercially available as Wingstay antioxidants). The antioxidant may be added to the latex composition/formulation as a 20-80 wt. % dispersion (e.g., 50% or any other percentage or range of percentages therein) in water, in an amount providing 0.1-3 phr of the antioxidant.

Release agents may include calcium carbonate and certain metal soaps. The metal soaps may include alkali, alkaline earth and late transition metal (e.g., Group 10-12 of the Periodic Table) salts of fatty acids, such as sodium, zinc and calcium salts of C₁₂-C₂₄ saturated, monounsaturated or polyunsaturated carboxylic acids (e.g., zinc or calcium stearate). The calcium carbonate may have a particle size of ≤1 micron and/or a BET surface area of ≥5 m²/g. The release agent may be added as a solution or emulsion containing 10-80 wt. % (e.g., 20-50 wt. %, or any wt. % or wt. % range therein) of the release agent, in an amount providing 0.1-10 phr of the release agent. Alternatively, a non-hydrocarbon oil, such as a silicone oil, can be added to the latex composition/formulation or sprayed (or otherwise applied) in a thin coat onto the mold prior to immersion in the latex composition/formulation.

To facilitate thorough mixing, all dispersions may be ground for 2-8 hours in an attrition mill using 1-5 mm (e.g., 3 mm) diameter metal oxide (e.g., zirconium oxide) or ceramic (e.g., glass) balls. Optionally, a dispersion agent (e.g., an alkali metal [such as sodium] salt of a naphthalene-sulfonic acid or condensate thereof, such as a TAMOL dispersant, commercially available from BASF, Ludwigshafen, Germany) may be added in an amount of 1% to 3% by weight. The dispersions may then be mixed with the latex and agitated (e.g., mechanically stirred) for a further 12 to 15 hours. The thus-formed latex formulations were then aged for 12-48 hours (e.g., 24 hours) and agitated (e.g., stirred) again for 10 minutes. The glove mold is then dipped into the latex. The mold with the glove is then withdrawn and dipped into a rinse solution (e.g., as described herein) for 20 minutes. It is oven-dried for 5-60 (e.g., 20) minutes at 80-150° C. (e.g., 100° C.). After dusting with zinc stearate or calcium carbonate, the glove is removed, inspected, and packaged for sale or shipment.

The following examples illustrate recipes for latex gloves and with high strength and anti-microbial properties.

### Example 1—Latex Gloves

The following table lists recipes for latex gloves.

TABLE 1
Latex Glove Compositions

| Recipe | 1A | 1B | 1C | 1D | 1E | 1F |
|---|---|---|---|---|---|---|
| Concentrated natural | 167 | 167 | 167 | 167 | 167 | 167 |
| latex 60% DRC | | | | | | |
| Potassium laurate 20% | 3.0 | 3.0 | 3.0 | 3.0 | 3.0 | 3.0 |
| 50% sulfur dispersion | 2.0 | 2.0 | 2.0 | 2.0 | 2.0 | 2.0 |
| 50% ZDEC dispersion | 0.8 | 0.8 | 0.8 | 0.8 | 0.8 | 0.8 |
| 50% ZMBT dispersion | 0.8 | 0.8 | 0.8 | 0.8 | 0.8 | 0.8 |
| 50% Wingstay dispersion | 2.0 | 2.0 | 2.0 | 2.0 | 2.0 | 2.0 |
| Wt. % CaCO₃ dispersion | 10.0 | 10.0 | 10.0 | 10.0 | 10.0 | 10.0 |
| 50% French process ZnO | 1.6 | 3.2 | — | — | — | — |
| dispersion | | | | | | |
| 50% ZnO HC dispersion | — | — | 1.6 | 3.2 | — | — |
| 50% ZnO NC236 | — | — | — | — | 1.6 | 3.2 |
| dispersion | | | | | | |
| Water | 85 | 85 | 85 | 85 | 85 | 85 |
| Total | 272.2 | 273.8 | 272.2 | 273.8 | 272.2 | 273.8 |

After dipping and curing at 100° C. in hot air for 20 minutes, we obtain the following properties:

TABLE 2
Latex gloves, original properties, Example 1

| Recipe | 1A | 1B | 1C | 1D | 1E | 1F |
|---|---|---|---|---|---|---|
| 300% Modulus, MPa | 1.29 | 1.57 | 1.14 | 1.45 | 1.54 | 1.75 |
| 500% Modulus, MPa | 2.96 | 4.32 | 3.49 | 4.23 | 3.74 | 5.23 |
| 700% Modulus, MPa | 14.8 | 17.1 | 13.76 | 16.57 | 18.13 | 21.32 |
| Tensile Strength, MPa | 32.6 | 32.63 | 31.31 | 32.96 | 32.16 | 33.8 |
| Elongation at break, % | 866.7 | 846.7 | 866.7 | 820.0 | 840 | 826.7 |
| Tear strength, kN/m | 46.9 | 42.64 | 43.90 | 53.6 | 47.33 | 53.52 |

After aging at 100° C. for 22 hours, we obtain the following properties:

TABLE 3
Latex gloves, aged properties, Example 1

| Recipe | 1A | 1B | 1C | 1D | 1E | 1F |
|---|---|---|---|---|---|---|
| 300% Modulus, MPa | 1.39 | 1.54 | 1.27 | 1.45 | 1.46 | 1.19 |
| 500% Modulus, MPa | 3.37 | 3.33 | 3.09 | 4.24 | 3.48 | 3.02 |
| 700% Modulus, MPa | 17.67 | — | 12.2 | 16.57 | 14.78 | 14.40 |
| Tensile Strength, MPa | 20.61 | 17.30 | 18.21 | 32.96 | 21.40 | 19.80 |
| Elongation at break, % | 746.7 | 626.7 | 766.7 | 820 | 746.7 | 746.7 |
| Tear strength, kN/m | 17.54 | 13.61 | 18.84 | 53.6 | 16.69 | 18.69 |

The only difference among the six recipes 1A-1F is the type and amount of activator(s). Surprisingly, the elasticity properties of latex gloves made from formulations containing the nanocomposite zinc oxide NC236 are much better than those of latex gloves made from formulations containing French process ZnO. The tensile strength, elongation at break, and tear strength are similar, but the amount of zinc in the formulation is lower, making these resulting surprising as well.

Using a Bacteria Science kit from EZ BioResearch (St. Louis, Mo. 63132), the ability of the above latex gloves to suppress growth of Staphylococcus aureus bacteria was determined. The antibacterial growth suppression ability of gloves made from the recipes 1A-1F above was, in order of greatest to least, 1F>1D≈1B>1C≈1A >1E. The zinc oxide content of the gloves made from recipe 1E is the smallest, at (0.6×1.6×0.5)=0.48 phr, while the gloves made from recipe 1A have 1.6×0.5=0.8 phr of zinc oxide (the “0.5” factor is included because the ZnO component was added as a 50% by weight dispersion). The gloves made from recipe 1F have a zinc oxide content of (0.6×3.2×0.5)=0.96 phr, but their antibacterial activity is higher than the gloves made from recipes 1D (containing nano zinc oxide) and 1B (containing French process zinc oxide), both of which contain (3.2×0.5)=1.6 phr of ZnO. The fact that the gloves made from recipe 1F have more antibacterial growth suppression activity than the gloves made from recipe 1D is totally unexpected, as both ZnO sources are in the form of nanoparticles, and the zinc oxide content of recipe 1D is much higher than recipe 1F.

### Example 2—Anti-Microbial Latex Gloves

The recipes in Example 1 were modified slightly by adding more activator (i.e., zinc oxide). The purpose is to make the glove stronger and more antimicrobial. The following table lists the modified recipes.

TABLE 4
Latex Glove Recipes

| Recipe | 2A | 2B | 2C | 2D | 2E | 2F | 2G |
|---|---|---|---|---|---|---|---|
| Concentrated | 167 | 167 | 167 | 167 | 167 | 167 | 167 |
| Natural latex | | | | | | | |
| Potassium laurate | 3.0 | 3.0 | 3.0 | 3.0 | 3.0 | 3.0 | 3.0 |
| 20% | | | | | | | |
| Sulfur, 50% | 2 | 2 | 2 | 2 | 2 | 2 | 2 |
| dispersion | | | | | | | |
| ZDEC, 50% | 0.8 | 0.8 | 0.8 | 0.8 | 0.8 | 0.8 | 0.8 |
| dispersion | | | | | | | |
| ZMBT, 50% | 0.8 | 0.8 | 0.8 | 0.8 | 0.8 | 0.8 | 0.8 |
| dispersion | | | | | | | |
| Wingstay, 50% | 2.0 | 2.0 | 2.0 | 2.0 | 2.0 | 2.0 | 2.0 |
| dispersion | | | | | | | |
| CaCO₃, 50% | 10.0 | 10.0 | 10.0 | 10.0 | 10.0 | 10.0 | 10.0 |
| dispersion | | | | | | | |
| French process ZnO, | 3.2 | 8 | — | — | — | — | — |
| 50% dispersion | | | | | | | |
| ZnO HC, 50% | — | — | 3.2 | 8 | — | — | — |
| dispersion | | | | | | | |
| ZnO NC236, 50% | — | — | — | — | 3.2 | 6.4 | 8 |
| dispersion | | | | | | | |
| Water | 85 | 85 | 85 | 85 | 85 | 85 | 85 |
| Total | 273.8 | 278.6 | 273.8 | 278.6 | 273.8 | 275 | 278.6 |

After dipping the molds in the above compositions and curing at 100° C. for 20 minutes, the obtained gloves had the properties shown in Table 5 below.

TABLE 5
Latex gloves, original properties, Example 2

| Recipe | 2A | 2B | 2C | 2D | 2E | 2F | 2G |
|---|---|---|---|---|---|---|---|
| 300% Modulus, MPa | 1.19 | 1.05 | 1.62 | 0.84 | 1.27 | 1.57 | 1.57 |
| 500% Modulus, MPa | 3.26 | 3.12 | 3.11 | 3.19 | 3.29 | 3.33 | 4.49 |
| 700% Modulus, MPa | 12.9 | 13.55 | 15.03 | 14.58 | 13.89 | 14.81 | 16.94 |
| Tensile Strength, MPa | 34.2 | 25.26 | 24.77 | 32.45 | 32.82 | 32.78 | 34.40 |
| Elongation at break, % | 886.7 | 813.3 | 833.3 | 846.7 | 873.3 | 833.3 | 840.0 |
| Tear strength, kN/m | 52.1 | 42.54 | 46.67 | 54.2 | 44.49 | 62.44 | 60.68 |

After aging at 100° C. for an additional 22 hours, the gloves had the following properties:

TABLE 6
Latex gloves, aged properties, Example 2

| Recipe | 2A | 2B | 2C | 2D | 2E | 2F | 2G |
|---|---|---|---|---|---|---|---|
| 300% Modulus, MPa | 1.19 | 1.20 | 1.08 | 0.83 | 1.00 | 1.18 | 1.02 |
| 500% Modulus, MPa | 3.39 | 3.55 | 3.09 | 3.40 | 3.00 | 3.49 | 3.56 |
| 700% Modulus, MPa | 14.83 | 14.91 | 14.31 | 15.38 | 14.37 | 16.03 | 15.14 |
| Tensile Strength, MPa | 27.72 | 25.04 | 18.62 | 24.25 | 22.46 | 28.89 | 25.28 |
| Elongation at break, % | 813.3 | 800.0 | 753.3 | 753.3 | 793.3 | 793 | 746.7 |
| Tear strength, kN/m | 21.38 | 17.01 | 19.09 | 18.59 | 17.07 | 19.98 | 18.89 |

The additional French process zinc oxide in recipe 2B rendered the gloves less elastic and more brittle, while the nanoparticulate zinc oxide in recipes 2C-2D and the nanocomposite zinc oxide in recipes 2E-2G generally did not. As greater amounts of zinc oxide provide greater antimicrobial activity, the amount(s) of nanocomposite zinc oxide (alone or combined with nanoparticulate zinc oxide) may be ideally maximized, as long as the latex gloves remain sufficiently elastic and flexible for commercial acceptance.

Using Escherichia coli (E. coli) bacteria instead of Staphylococcus aureus bacteria, we found the bacteria suppression ability to be in the following order: 2G>2F>2C>2E>2D>2A>2B with 2G being the most inhibiting to the growth of E. coli in nutrient agar. These results show again the present nanocomposite zinc oxide's superior antimicrobial property to French process ZnO and nano ZnO alone.

### CONCLUSION/SUMMARY

The present invention concerns latex and natural rubber compositions and formulations, latex or rubber products made using the same, and methods of preparing vulcanized latex/rubber and of making latex and natural rubber products having antibacterial, antifungal and/or antiviral properties. The compositions and formulations include at least one nanocomposite zinc oxide, which functions as (i) an accelerator and/or activator and (ii) an anti-microbial agent. When the nanocomposite zinc oxide is a nanocomposite zinc oxide that includes a calcium carbonate core material, the nanocomposite zinc oxide may also function as a dispersant (e.g., of the zinc oxide) and as an anti-aging agent. The present compositions and formulations do not render the latex or rubber products brittle or less elastic in comparison with latex and natural rubber compositions and formulations containing the same or higher dosages of French process zinc oxide (in place of the [nano]composite zinc oxide). The lower cost of and lower zinc discharge to the environment (e.g., as compared to ZnO) are additional advantages of the present compositions and formulations.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

1. A composition or formulation, comprising:
a) latex or natural rubber;
b) a vulcanization activator in an amount of 0.1-5 parts per hundred parts of the latex or natural rubber (phr); and
c) one or more accelerators and/or additional activators in an amount of 0.5-10 phr, wherein at least one of the one or more accelerators and/or additional activators comprises a nanoparticulate zinc oxide or a nanocomposite zinc oxide, the nanoparticulate zinc oxide has an average primary particle size of 100 nm or less, the nanocomposite zinc oxide comprises (i) a core material and/or particle and (ii) a zinc oxide and/or zinc carbonate coating thereon, containing from 1% to 80% by weight of zinc oxide and/or zinc carbonate, and both the core material and/or particle and the zinc oxide and/or zinc carbonate coating have an average primary particle size of 100 nm or less, or a particle size distribution in which ≥50% of the particles have a particle size of 100 nm or less.

2. The composition or formulation of claim 1, further comprising water in an amount of 50-100 phr.

3. The composition or formulation of claim 1, further comprising a release agent in an amount of 0.1-2 phr.

4. The composition or formulation of claim 1, further comprising an antioxidant in an amount of 0.1-3 phr.

5. The composition or formulation of claim 1, further comprising a stabilizer in an amount of 0.1-5 phr.

6. The composition or formulation of claim 1, comprising the nanocomposite zinc oxide, wherein the nanocomposite zinc oxide comprises coprecipitated nano zinc oxide-coated calcium carbonate having (i) an average particle size of 200 nm or less, (ii) a particle size distribution in which ≥50% of the particles have a primary particle size of 100 nm or less, or (iii) from 25 to about 80 weight percent zinc oxide.

7. A latex or rubber product made from the composition or formulation of claim 1.

8. The latex or rubber product of claim 7, having (i) a tensile strength of at least 30 MPa or a tear strength of at least 40 kN/m, and (ii) an antimicrobial activity greater than that of an otherwise identical latex or rubber product made from an otherwise identical composition or formulation substituting an identical amount of a French process zinc oxide for the nanoparticulate zinc oxide and the nanocomposite zinc oxide.

9. The latex or rubber product of claim 8, having a modulus of elasticity at 300% or greater elongation that is greater than that of the otherwise identical latex or rubber product made from the otherwise identical composition or formulation substituting the identical amount of the French process zinc oxide for the nanoparticulate zinc oxide and the nanocomposite zinc oxide.

10. (canceled)

11. The latex or rubber product of claim 7, comprising a glove, condom, or balloon having a thickness of 0.05-0.5 mm.

12. The latex or rubber product of claim 7, further comprising a fabric.

13. The latex or rubber product of claim 7, comprising a foam rubber product.

14. A method of preparing a vulcanized latex or rubber, comprising:
a) mixing a source of the latex or rubber with 0.1-5 parts per hundred parts of the latex or natural rubber (phr) of a vulcanization activator or vulcanizing agent, 0.5-10 phr of one or more nanoparticulate zinc oxides or nanocomposite zinc oxides, and optionally one or more additional accelerators and/or activators, pigments, stabilizers, release agents, and/or antioxidants in a tank or vessel to form a latex or rubber formulation, wherein the nanoparticulate zinc oxide has an average primary particle size of 100 nm or less, the nanocomposite zinc oxide comprises (i) a core material and/or particle and (ii) a zinc oxide and/or zinc carbonate coating thereon, containing from 1% to 80% by weight of zinc oxide and/or zinc carbonate, and both the core material and/or particle and the zinc oxide and/or zinc carbonate coating have an average primary particle size of 100 nm or less, or a particle size distribution in which >50% of the particles have a particle size of 100 nm or less;
b) dipping or at least partially immersing one or more molds or formers in the latex or rubber formulation to form a latex or rubber coating on the one or more molds or formers; and
c) curing the latex or rubber coating to vulcanize the latex or rubber coating.

15. The method of claim 14, further comprising, prior to or during curing the latex or rubber coating, drying the latex or rubber coating on the one or more molds or formers.

16. The method of claim 14, wherein curing the latex or rubber coating comprises heating the curable rubber composition at a temperature of at least 80° C. for at least 10 minutes.

17. The method of claim 14, further comprising:
a) washing the one or more molds or formers in water and/or an aqueous chlorine-based solution prior to dipping or at least partially immersing the one or more molds or formers in the latex or rubber formulation; and/or
b) prior to curing the latex or rubber coating, immersing the latex or rubber coating on the one or more molds or formers in a mixture of water and a chlorine source at a temperature and for a length of time sufficient to remove residual latex proteins and non-rubber chemicals and/or reduce the severity of any allergic reactions to the latex.

18. A method of making a latex or rubber product, comprising the method of claim 14 and releasing the cured latex or rubber coating from the one or more molds or formers to produce the latex or rubber product.

19. The method of claim 18, wherein the latex or rubber product has an elongation at break over 700%.

20. The method of claim 18, wherein the latex or rubber product has a tensile strength, a tear strength, and a modulus of elasticity that are equal to or better than an otherwise identical latex or rubber product made from an otherwise identical latex or rubber formulation containing an amount of French press zinc oxide identical to an amount of the one or more the nanoparticulate zinc oxides and nanocomposite zinc oxides in the latex or rubber formulation.

21. The composition or formulation of claim 1, comprising the nanoparticulate zinc oxide, in an amount of 0.5-4 phr of the latex or natural rubber.
